(19) United States
(12) Patent Application Publication
(10) Pub. No.: US 2008/0026987 A1
(43) Pub. Date: Jan. 31, 2008

(54) Use of Liver-Selective Glucokinase Activators

(75) Inventors: Peter Mackay, Charlottenlund (DK); James Mackay, Charlottenlund (DK); Carmen Valcarce-Lopez, Limhamn (SE); Thora Brynja Bodvarsdottir, Vedbaek (DK); Keld Fosgerau, Vanlose (DK); Marianne Olholm Larsen, Naerum (DK); Per O.G. Arkhammar, Helsingborg (DK); Philip Wahl, Hillerod (DK)

Correspondence Address: NOVO NORDISK, INC.;PATENT DEPARTMENT, 100 COLLEGE ROAD WEST, PRINCETON, NJ, 08540, US

(73) Assignee: Novo Nordisk A/S, Bagsvaerd (DK)

(21) Appl. No.: 11/629,711

(22) PCT Filed: Jun. 17, 2005
(86) PCT No.: PCT/EP05/52818

§ 371 Date: Oct. 5, 2007

(30) Foreign Application Priority Data
Jun. 17, 2004 (DK) PA 2004 00939

(51) Int. Cl. A61K 38/28; A61K 31/426; A61K 31/498; A61P 31/10; A61K 31/502; A61K 31/506

(52) U.S. Cl. 514/4; 514/252.1; 514/255.01; 514/256; 514/365

(57) ABSTRACT

The present invention provides a method of normalizing blood glucose levels in mammals utilizing liver-selective glucokinase activators. The present invention also provides a method of increasing liver metabolism and decreasing apoptosis independent of glucose normalization or hyperglycemic conditions.

### FIELD OF THE INVENTION

The present invention generally relates to a method of normalizing blood glucose levels in mammals utilizing liver-selective glucokinase activators. The present invention also relates to a method of increasing liver metabolism and decreasing apoptosis independent of glucose normalization or hyperglycemic conditions.

### BACKGROUND OF THE INVENTION

Glucokinase (GK) is one of four hexokinases that are found in mammals. The hexokinases catalyze the first step in the metabolism of glucose, the conversion of glucose to glucose-6-phosphate. GK plays an essential role in blood glucose homeostasis. GK catalyses glucose phosphorylation, and is the rate-limiting reaction for glycolysis in liver parenchymal cells and pancreatic β-cells. In liver, GK determines the rates of both glucose uptake and glycogen synthesis, and it is also thought to be essential for the regulation of various glucose-responsive genes. In the pancreatic β-cells, GK determines glucose utilization and thus is necessary for glucose-stimulated insulin secretion. GK is also expressed in a population of neurons in the hypothalamus (where it may be involved in feeding behavior), and in the gut (where it may contribute to the secretion of enteroincretins, such as GLP-1). However, the functional importance of GK in these tissues has not yet been defined.

Numerous studies have been done to determine the effect of either increased, or diminished GK gene expression on blood glucose homeostasis. These studies revealed a reciprocal relationship between GK gene copy number and blood glucose concentration. Based on these studies, strategies directed toward increasing the expression, or the activity of GK have been proposed as a novel therapeutic approach for the treatment of type 2 diabetes, as they are thought to result in an improvement of glucose homeostasis.

In humans, GK mutations have been associated with maturity onset diabetes of the young type 2 (MODY2). Other examples of GK mutations are discussed in Gloyn, A. L. (2003) Hum Mutat 22, 353-362. The literature also suggests that a decrease in GK activity, or expression, may contribute to postprandial hyperglycemia in people with type 2 diabetes.

Many patent applications describe a class of chemical compounds termed glucokinase activators, which increase insulin secretion and are thought useful for the treatment of type 2 diabetes. However, the risk for hypoglycemia, resulting from an increase in insulin secretion in response to GK activation of the pancreatic β-cell, remains an issue with the use of these compounds. The risk of developing hypoglycemia under these conditions is illustrated by the fact that patients that carry GK activating mutations that alter the affinity of GK for glucose, both in the pancreas and in the liver, suffer from hyperinsulinism and hypoglycemia.

The present invention describes a method wherein liver GK is selectively activated, thereby causing a reduction of blood glucose without significant risk of hypoglycemia.

### SUMMARY OF THE INVENTION

The present invention provides a method for glucose normalization without a significant risk of hypoglycemia.

The present invention also provides a method for the treatment of type I diabetes.

The present invention also provides a method for the prevention of microvascular diseases, including but not limited to nephropathy, neuropathy, retinopathy, and the like, wherein the method is carried out alone or in combination with other therapeutic agents, wherein the method brings about blood glucose normalization.

The present invention also provides a method for the prevention of development of diabetes in risk populations (including but not limited to individuals having Impaired Glucose Tolerance, GDM, PCOS, Metabolic Syndrome), wherein the method brings about blood glucose normalization.

The present invention also provides a method for the prevention of development of macrovascular diseases in risk populations (including but not limited to individuals having Impaired Glucose Tolerance, GDM, Metabolic syndrome) alone or in combination with lipid lowering drugs for example, LPL activators, HSL inhibitors, estatins, fibrates, PPAR α agonists, PPAR δ agonists, and the like, wherein the method brings about blood glucose normalization. Macrovascular diseases include but are not limited to atherosclerotic cardiovascular disease, coronary artery disease (CAD), cerebrovascular disease, peripheral vascular disease, heart failure, and hypertension.

The present invention also provides a method for the treatment of pathological conditions associated with low GK activity, for example, MODY2, and Persistent Neonatal Diabetes Mellitus due to GK homozygous mutations.

The present invention also provides a method for the preservation of beta-cell mass and function, effected by blood glucose normalization.

The present invention also provides a method for increasing beta cell mass and function.

The present invention also provides for anti-apoptotic/protective effects of glucokinase activators in relation to IAPP induced beta cell death.

The present invention also provides a method for the protective effect on amyloid beta peptide induced cell death.

The present invention also provides a method for veterinary use for all indications that could benefit from blood glucose normalization, including administration as a food additive.

The present invention also provides a method for the treatment of hepatic conditions that benefit from blood glucose normalization.

The present invention also provides a method for the treatment of hepatic conditions that benefit from improved liver function and/or antiapoptotic effect, for example cirrhotic liver, nonalcoholic steatohepatitis (nonalcoholic fatty liver disease), alcoholic steatohepatitis (alcoholic fatty liver disease), macrovesicular fatty liver, microvesicular fatty liver, alcoholic hepatitis, amyloidosis, and alcoholic cirrhosis.

The present invention also provides a method for the treatment of hyperglycemic conditions that result from critical illness, or are a consequence of diverse therapies, for example HIV-treatment.

The present invention also provides a method for the treatment of hepatic conditions that result from critical illness like cancer, or are a consequence of diverse therapies, for example cancer therapy, HIV-treatment.

The present invention also provides a method for the treatment of type 1 diabetes in combination with short-acting insulin, long-acting insulin, or a mix of short- and long-acting insulin, in all types of formulations and administration regimens (including but not limited to peroral, subcutaneous injection, inhalation, patches, pumps, and the like).

The present invention also provides a method of treatment adjuvant to insulin in insulin-requiring diabetes type 2 or as replacement for insulin in said therapy.

The present invention also provides a method for the treatment of lipodistrophy.

The present invention also provides a method for the treatment of hyperglycemia in relation to severe physical stress without signs of liver failure, such as multiple trauma, diabetic ketoacidosis, and the like.

The present invention also provides a method of preventing type 1 diabetes.

The present invention also provides a method of preserving and/or increasing beta-cell mass and function in patients having undergone pancreatic islet transplantation.

The present invention also provides a method of improving glucose control during and after surgery.

The present invention also provides a method of improving liver function and/or survival in patients undergoing liver transplantation, wherein the administration may occur before, during or after transplantation, or any combination thereof.

The present invention also provides a method of obtaining blood glucose normalization.

The present invention also provides a method of preventing or ameliorating diabetic late complications.

The present invention also provides a method of treating type 1 or 2 diabetes, wherein the treatment does not result in a weight gain.

The present invention also provides a method of preventing diabetic ketoacidosis.

### DETAILED DESCRIPTION OF THE INVENTION

Glucokinase has two main distinctive characteristics: (1) its expression, which is limited to tissues that require glucose-sensing (mainly liver and pancreatic β-cells), and (2) its sigmoidal saturation curve, which has a value of S_{0.5} for glucose that is much higher (8-12 mM) than that of the other members of the hexokinase family. Due to these kinetic characteristics, changes in serum glucose levels are paralleled by changes in glucose metabolism in liver, which in turn regulate the balance between hepatic glucose output (HGO) and glucose consumption.

Tissue-specific differences between GK regulation in liver and pancreatic β-cells have been observed. In liver, GK gene transcription is stimulated by insulin and inhibited by glucagon. Importantly, in the liver the activity of GK is also modulated by a glucokinase regulatory protein (GKRP), which binds and inhibits GK competitively with respect to glucose. Under basal glucose conditions (˜5.5 mM) hepatic GK is bound largely to GKRP and it is located in the nucleus of the hepatocytes. However, after exposure to either high glucose (10-30 mM) or fructose (50 μM to 1 mM), GK is released from the GKRP and exits the nucleus in an unbound state ready to phosphorylate glucose. In the islets, GK gene expression is thought to be largely constitutive, although glucose modulates islets GK content, probably by directly affecting the half-life of the enzyme since GKRPs have not been shown on pancreatic β-cells. Experiments in transgenic animals over-expressing GK in the liver clearly revealed the reciprocal relationship between GK gene copy number (GK activity), and blood glucose concentration, without observed changes in insulin secretion.

The methods of the present invention utilize liver-selective glucokinase activators, as described for example in WO 2004 002481, the contents of which are incorporated by reference herein in its entirety. The use of liver-selective GK activators (GK activators which increase glucose utilization in the liver without inducing an increase in insulin secretion in response to glucose), will have a very low risk of inducing hypoglycemia. Even in the case where the activators alter the kinetics of the hepatic GK, the GKRP will inhibit GK at glucose concentrations lower than the basal levels. This advantage is greater in the situation where GK activators alter the sigmoidal saturation curve of the GK kinetics, and increase its affinity for glucose.

Selective activation of the glucokinase in the liver increases hepatic glucose utilization in a glucose dependent manner. Surprisingly, this increase is enough to normalize blood glucose levels in diabetic animals without the need of an increase in insulin secretion. Selective activation of the glucokinase in the liver will be an optimal mechanism to control glycemia. It will present major advantages with respect to current treatments: (i) it will decrease the stress on the beta-cells, that is it will protect the beta cell mass/function and as a consequence delay/stop the progression of the disease; (ii) normalization of blood glucose during the most of the day will reduce significantly Hemoglobin A_{1C} and it will prevent or ameliorate the onset of late diabetic complications; (iii) since the mechanism is glucose dependent the risk of hypoglycemic events will be negligible; (iv) the mode of action does not involve increase in plasma insulin levels, thus, the side effects derived from an excess of insulin, such as increase in fat mass and body weight, hypoglycemia, macrovascular disease, and dyslipidemia, will be irrelevant.

Recent biochemical findings showed that liver glucokinase is found in a complex with a key regulator of apoptosis the protein BAD supporting a link between glucose metabolism and apoptosis.

An increase in hepatic glycolysis induced by activators of glucokinase will improve liver conditions that involved massive apoptosis, such as acute liver failure or cirrhotic liver. Liver health depends on the efficient removal of unwanted cells, such as aged or virus-infected cells, mainly through apoptosis. In a physiologic setting, new cells generated by mitosis replace those that are eliminated, ensuring organ homeostasis. An alteration in this balance between cell death and proliferation can cause liver diseases such as cancer or hepatitis, depending on whether the balance is tilted toward proliferation or apoptosis. Excessive apoptosis after acute injury results in destruction of extensive areas of liver tissue, whereas persistent, moderately high apoptosis leads to fibrosis and perhaps cirrhosis. Given the poor prognosis and high mortality rates associated with most liver diseases, a therapy to reduce apoptosis in this organ has important clinical applications. Liver selective glucokinase activators will provide a liver-targeted antiapoptotic therapy. Such hepatoprotective therapy could be useful in acute liver injury to save lives, and in chronic liver injury to minimize hepatic fibrosis.

Hepatic conditions benefiting from the present invention include, but are not limited, to cirrhotic liver, acute liver failure, liver failure due to other drugs treatment, steatosis, nonalcoholic steatohepatitis (nonalcoholic fatty liver disease), alcoholic steatohepatitis (alcoholic fatty liver disease), macrovesicular fatty liver, microvesicular fatty liver, alcoholic hepatitis, alcoholic cirrhosis

While the invention has been described and illustrated with reference to certain preferred embodiments thereof, those skilled in the art will appreciate that various changes, modifications and substitutions can be made therein without departing from the spirit and scope of the present invention. For example, effective dosages other than the preferred dosages as set forth herein may be applicable as a consequence of variations in the responsiveness of the mammal being treated for glucokinase-deficiency mediated disease(s). Likewise, the specific pharmacological responses observed may vary according to and depending on the particular active compound selected or whether there are present pharmaceutical carriers, as well as the type of formulation and mode of administration employed, and such expected variations or differences in the results are contemplated in accordance with the objects and practices of the present invention.

The term liver-selective glucokinase activator in this context means a compound which increases glucose utilization in the liver without inducing any significant increase in insulin secretion in response to glucose.

In another embodiment, a liver-selective glucokinase activator can be viewed as a compound which shows a significantly higher activity in isolated hepatocytes compared to the activity of the compound in Ins-1 cells.

In another embodiment, a liver-selective glucokinase activator can be viewed as a compound which shows a significantly higher activity in isolated hepatocytes measured as described in the Glucokinase Activity Assay (II) compared to the activity of the compound in Ins-1 cells measured as described in the Glucokinase Activity Assay (III).

In another embodiment, a liver-selective glucokinase activator can be viewed as a compound which shows an activity in isolated hepatocytes measured as described in the Glucokinase Activity Assay (II) which activity is at least 1.1 fold higher, such as at least 1.2 fold higher, for instance at least 1.3 fold higher, such as at least 1.4 fold higher, for instance 1.5 fold higher, such as at least 1.6 fold higher, for instance at least 1.7 fold higher, such as at least 1.8 fold higher, for instance at least 1.9 fold higher, such as at least 2.0 fold higher, for instance at least a 3.0 fold higher, such as at least a 4.0 fold higher, for instance at least 5.0 fold higher, such as at least 10 fold higher than the activity of the compound in Ins-1 cells measured as described in the Glucokinase Activity Assay (III).

In another embodiment, a liver-selective glucokinase activator can be viewed as a compound which shows no activity in the Ins-1 cells measured as described in the Glucokinase Activity Assay (III).

The term normoglycemia in this context has the meaning normally understood by the person skilled in the art, e.g. blood glucose levels below the definition of hyperglycemia and above those that will be considered as hypoglycemia, both as given by the American Diabetes Association or World Health Organization.

Alternatively hypoglycemia can be understood as that level of blood glucose at which the counter-regulatory mechanisms get switched on.

The term treatment in this context means the management and care of a patient for the purpose of combating a disease, disorder or condition. The term is intended to include the full spectrum of treatments for a given disorder from which the patient is suffering, such as the delaying of the progression of the disease, disorder or condition, the alleviation or relief of symptoms and complications, the prevention of the disease and/or the cure or elimination of the disease, disorder or condition. The patient to be treated is preferably a mammal, in particular a human being.

Thus, the present invention provides a method for the treatment of glucokinase-deficiency mediated conditions/diseases, or conditions benefiting from an increase in glucokinase activity, comprising administering to a subject in need of such treatment a liver-selective glucokinase activator or pharmaceutical composition thereof, wherein blood glucose normalization occurs with reduced risk of hypoglycemia.

In another embodiment the invention provides a method wherein the condition/disease as mentioned above is type I diabetes.

In another embodiment the invention provides a method wherein the liver-selective glucokinase activator is administered in combination with insulin, short-acting insulin, long-acting insulin, or a combination thereof.

In another embodiment the invention provides a method wherein the glucokinase-deficiency mediated condition/disease is caused by a glucokinase mutation.

In another embodiment the invention provides a method wherein the glucokinase-deficiency mediated condition/disease is Maturity-Onset Diabetes of the Young, Neonatal Diabetes Mellitus, or Persistent Neonatal Diabetes Mellitus.

In another embodiment the invention provides a method for preventing the development of diabetes in subjects exhibiting symptoms of Impaired Glucose Tolerance, Gestational Diabetes Mellitus, Polycystic Ovarian Syndrome, Cushings syndrome or Metabolic Syndrome comprising administering to a subject in need of such treatment a liver-selective glucokinase activator or pharmaceutical composition thereof, wherein blood glucose normalization occurs with reduced risk of hypoglycemia.

In another embodiment the invention provides a method for preventing microvascular diseases comprising administering to a subject in need of such treatment a liver-selective glucokinase activator or pharmaceutical composition thereof.

In another embodiment the invention provides a method for preventing macrovascular diseases in subjects exhibiting symptoms of Impaired Glucose Tolerance, Gestational Diabetes Mellitus, or Metabolic Syndrome, comprising administering to a subject in need of such treatment a liver-selective glucokinase activator or pharmaceutical composition thereof, alone or in combination with lipid-lowering drugs, wherein blood glucose normalization occurs with reduced risk of hypoglycemia.

In another embodiment the invention provides a method for the preservation of beta-cell mass and function comprising administering to a subject in need of such treatment a liver-selective glucokinase activator or pharmaceutical composition thereof, wherein blood glucose normalization occurs with reduced risk of hypoglycemia.

In another embodiment the invention provides a method for preventing amyloid beta peptide induced cell death comprising administering to a subject in need of such treatment a liver-selective glucokinase activator or pharmaceutical composition thereof, wherein blood glucose normalization occurs with reduced risk of hypoglycemia.

In another embodiment the invention provides a method wherein the subject is a veterinary subject.

In another embodiment the invention provides a method wherein the liver-selective glucokinase activator is administered as a food additive.

In another embodiment the invention provides a method for the treatment of hepatic conditions benefiting from blood glucose normalization comprising administering to a subject in need of such treatment a liver-selective glucokinase activator or pharmaceutical composition thereof, wherein blood glucose normalization occurs with reduced risk of hypoglycemia.

In another embodiment the invention provides a method for the treatment of hepatic conditions benefiting from improved liver function comprising administering to a subject in need of such treatment a liver-selective glucokinase activator or pharmaceutical composition thereof.

In another embodiment the invention provides a method for the treatment of hyperglycemic conditions that result from critical illness, or as a consequence of therapeutic intervention comprising administering to a subject in need of such treatment a liver-selective glucokinase activator or pharmaceutical composition thereof, wherein blood glucose normalization occurs with reduced risk of hypoglycemia.

In another embodiment the invention provides a method for the treatment of hepatic conditions that result from critical illness like cancer, or are a consequence of therapy, for example cancer therapy or HIV-treatment, comprising administering to a subject in need of such treatment a liver-selective glucokinase activator or pharmaceutical composition thereof.

In another embodiment the invention provides a method of treatment adjuvant to insulin in insulin-requiring diabetes type 2, or as replacement for insulin comprising administering to a subject in need of such treatment a liver-selective glucokinase activator or pharmaceutical composition thereof, wherein blood glucose normalization occurs with reduced risk of hypoglycemia.

In another embodiment the invention provides a method for the treatment of lipodistrophy comprising administering to a subject in need of such treatment a liver-selective glucokinase activator or pharmaceutical composition thereof, wherein blood glucose normalization occurs with reduced risk of hypoglycemia.

In another embodiment the invention provides a method for the treatment of hyperglycemia resulting from severe physical stress without signs of liver failure comprising administering to a subject in need of such treatment a liver-selective glucokinase activator or pharmaceutical composition thereof, wherein blood glucose normalization occurs with reduced risk of hypoglycemia.

In another embodiment the invention provides a method wherein the severe physical stress is multiple trauma, or diabetic ketoacidosis.

In another embodiment the invention provides a method for preventing apoptotic liver damage comprising administering to a subject in need of such treatment a liver-selective glucokinase activator or pharmaceutical composition thereof.

In another embodiment the invention provides a method for preventing hypoglycemia comprising administering to a subject in need of such treatment a liver-selective glucokinase activator or pharmaceutical composition thereof, wherein blood glucose normalization occurs with reduced risk of hypoglycemia.

In another embodiment the invention provides a method for increasing beta-cell mass and function comprising administering to a subject in need of such treatment a liver-selective glucokinase activator or pharmaceutical composition thereof, wherein blood glucose normalization occurs with reduced risk of hypoglycemia.

In another embodiment the invention provides a method of preventing type 1 diabetes comprising administering to a subject in need of such treatment a liver-selective glucokinase activator or pharmaceutical composition thereof, wherein blood glucose normalization occurs with reduced risk of hypoglycemia.

In another embodiment the invention provides a method of preserving and/or increasing beta-cell mass and function in patients having undergone pancreatic islet transplantation comprising administering to a subject in need of such treatment a liver-selective glucokinase activator or pharmaceutical composition thereof.

In another embodiment the invention provides a method of improving glucose control during and after surgery comprising administering to a subject in need of such treatment a liver-selective glucokinase activator or pharmaceutical composition thereof.

In another embodiment the invention provides a method of improving liver function and/or survival in patients undergoing liver transplantation comprising administering to a subject in need of such treatment a liver-selective glucokinase activator or pharmaceutical composition thereof.

In another embodiment the invention provides a method according to claim 26 wherein the administration occurs before, during or after transplantation, or any combination thereof.

In another embodiment the invention provides a method of obtaining blood glucose normalization comprising administering to a subject in need of such treatment a liver-selective glucokinase activator or pharmaceutical composition thereof, wherein blood glucose normalization occurs with reduced risk of hypoglycemia.

In another embodiment the invention provides a method of preventing or ameliorating diabetic late complications comprising administering to a subject in need of such treatment a liver-selective glucokinase activator or pharmaceutical composition thereof.

In another embodiment the invention provides a method of treating type 1 or 2 diabetes comprising administering to a subject in need of such treatment a liver-selective glucokinase activator or pharmaceutical composition thereof, wherein the treatment does not result in a weight gain.

In another embodiment the invention provides a method of preventing diabetic ketoacidosis comprising administering to a subject in need of such treatment a liver-selective glucokinase activator or pharmaceutical composition thereof.

Dosage and Formulation

A suitable liver-selective glucokinase activator can be administered to patients to treat glucokinase-deficiency mediated conditions/diseases, or conditions benefiting from an increase in Glucokinase activity as the compound alone and/or mixed with an acceptable carrier in the form of pharmaceutical formulations. Those skilled in the art of treating glucokinase-deficiency mediated conditions/diseases or conditions benefiting from an increase in Glucokinase activity can easily determine the dosage and route of administration of the compound to mammals, including humans, in need of such treatment. The route of administration may include but is not limited to oral, intraoral, rectal, transdermal, buccal, intranasal, pulmonary, subcutaneous, intramuscular, intradermal, sublingual, intracolonic, intraoccular, intravenous, or intestinal administration. The compound is formulated according to the route of administration based on acceptable pharmacy practice (Fingl et al., in The Pharmacological Basis of Therapeutics, Ch. 1, p. 1, 1975; Remington's Pharmaceutical Sciences, 18^{th} ed., Mack Publishing Co, Easton, Pa., 1990).

The pharmaceutically acceptable liver-selective glucokinase activator composition of the present invention can be administered in multiple dosage forms such as tablets, capsules (each of which includes sustained release or timed release formulations), pills, powders, granules, elixirs, in situ gels, microspheres, crystalline complexes, liposomes, micro-emulsions, tinctures, suspensions, syrups, aerosol sprays and emulsions. The composition of the present invention can also be administered in oral, intravenous (bolus or infusion), intraperitoneal, subcutaneous, transdermally or intramuscular form, all using dosage forms well known to those of ordinary skill in the pharmaceutical arts. The composition may be administered alone, but generally will be administered with a pharmaceutical carrier selected on the basis of the chosen route of administration and standard pharmaceutical practice.

The dosage regimen for the composition of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. A physician or veterinarian can determine and prescribe the effective amount of the drug required to prevent, counter, or arrest the progress of the disease state.

By way of general guidance, the daily oral dosage of the active ingredient, when used for the indicated effects, will range between about 0.001 to 1000 mg/kg of body weight, preferably between about 0.01 to 100 mg/kg of body weight per day, and most preferably between about 0.6 to 20 mg/kg/day. Intravenously, the daily dosage of the active ingredient when used for the indicated effects will range between 0.001 ng to 100.0 ng per min/per Kg of body weight during a constant rate infusion. Such constant intravenous infusion can be preferably administered at a rate of 0.01 ng to 50 ng per mm per Kg body weight and most preferably at 0.1 ng to 10.0 mg per mm per Kg body weight. The composition of this invention may be administered in a single daily dose, or the total daily dosage may be administered in divided doses of two, three, or four times daily. The composition of this invention may also be administered by a depot formulation that will allow sustained release of the drug over a period of days/weeks/months as desired.

The composition of this invention can be administered in intranasal form via topical use of suitable intranasal vehicles, or via transdermal routes, using transdermal skin patches. When administered in the form of a transdermal delivery system, the dosage administration will, of course, be continuous rather than intermittent throughout the dosage regimen. The composition is typically administered in a mixture with suitable pharmaceutical diluents, excipients, or carriers (collectively referred to herein as pharmaceutical carriers) suitably selected with respect to the intended form of administration, that is, oral tablets, capsules, elixirs, aerosol sprays generated with or without propellant and syrups, and consistent with conventional pharmaceutical practices.

For instance, for oral administration in the form of a tablet or capsule, the active drug component can be combined with an oral, non-toxic, pharmaceutically acceptable, inert carrier such as but not limited to, lactose, starch, sucrose, glucose, methyl cellulose, magnesium stearate, dicalcium phosphate, calcium sulfate, mannitol, and sorbitol; for oral administration in liquid form, the oral drug components can be combined with any oral, non-toxic, pharmaceutically acceptable inert carrier such as, but not limited to, ethanol, glycerol, and water. Moreover, when desired or necessary, suitable binders, lubricants, disintegrating agents, and coloring agents can also be incorporated into the mixture. Suitable binders include, but not limited to, starch, gelatin, natural sugars such as, but not limited to, glucose or beta-lactose, corn sweeteners, natural and synthetic gums such as acacia, tragacanth, or sodium alginate, carboxymethylcellulose, polyethylene glycol, and waxes. Lubricants used in these dosage forms include, but are not limited to, sodium oleate, sodium stearate, magnesium stearate, sodium benzoate, sodium acetate, and sodium chloride. Disintegrants include, but are not limited to, starch, methyl cellulose, agar, bentonite, and xanthan gum.

The composition of the present invention may also be administered in the form of mixed micellar or liposome delivery systems, such as small unilamellar vesicles, large unilamellar vesicles, and multilamellar vesicles. Liposomes can be formed from a variety of phospholipids, such as cholesterol, stearylamine, or phosphatidyicholines. Permeation enhancers may be added to enhance drug absorption.

Since prodrugs are known to enhance numerous desirable qualities of pharmaceuticals (i.e., solubility, bioavailability, manufacturing, etc.) the compounds of the present invention may be delivered in prodrug form. Thus, the present invention is intended to cover prodrugs of the presently claimed compounds, methods of delivering the same and compositions containing the same.

The compositions of the present invention may also be coupled with soluble polymers as targetable drug carriers. Such polymers can include polyvinyl-pyrrolidone, pyran copolymer, polyhydroxypropyl-methacrylamide-phenol, polyhydroxyethylaspartamidephenol, or polyethyleneoxidepolylysine substituted with palmitoyl residues. Furthermore, the composition of the present invention may be combined with a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Dosage forms (pharmaceutical compositions) suitable for administration may contain from about 0.1 milligram to about 500 milligrams of active ingredient per dosage unit. In these pharmaceutical compositions the active ingredient will ordinarily be present in an amount of about 0.5-95% by weight based on the total weight of the composition. Gelatin capsules may contain the active ingredient and powdered carriers, such as lactose, starch, cellulose derivative, magnesium stearate, and stearic acid. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance.

In general, water, a suitable oil, saline, aqueous dextrose (glucose), and related sugar solutions and glycols such as propylene glycol or polyethylene glycols are suitable carriers for parenteral solutions. Solution for parenteral administration preferably contains a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol.

Suitable pharmaceutical carriers are described in Remington: The Science and Practice of Pharmacy, Nineteenth Edition, Mack Publishing Company, 1995, a standard reference text in this field Representative useful pharmaceutical dosage forms for administration of the compound of this invention can be illustrated as follows:

Capsules

A large number of unit capsules can be prepared by filling standard two-piece hard gelatin capsules with 100 milligrams of powdered active ingredient, 150 milligrams of lactose, 50 milligrams of cellulose, and 6 milligrams magnesium stearate.

Soft Gelatin Capsules

A mixture of active ingredient in a digestible oil such as soybean oil, cottonseed oil or olive oil may be prepared and injected by means of a positive displacement pump into gelatin to form soft gelatin capsules containing 100 milligrams of the active ingredient. The capsules should be washed and dried.

Tablets

Tablets may be prepared by conventional procedures so that the dosage unit, for example is 100 milligrams of active ingredient, 0.2 milligrams of colloidal silicon dioxide, 5 milligrams of magnesium stearate, 275 milligrams of microcrystalline cellulose, 11 milligrams of starch and 98.8 milligrams of lactose. Appropriate coatings may be applied to increase palatability or delay absorption.

Injectable

A parenteral composition suitable for administration by injection may be prepared by stirring for example, 1.5% by weight of active ingredient in 10% by volume propylene glycol and water. The solution should be made isotonic with sodium chloride and sterilized.

Suspension

An aqueous suspension can be prepared for oral and/or parenteral administration so that, for example, each 5 mL contains 100 mg of finely divided active ingredient, 20 mg of sodium carboxymethyl cellulose, 5 mg of sodium benzoate, 1.0 g of sorbitol solution, U.S.P., and 0.025 mL of vanillin or other palatable flavoring.

Biodegradable Microparticles

A sustained-release parenteral composition suitable for administration by injection may be prepared, for example, by dissolving a suitable biodegradable polymer in a solvent, adding to the polymer solution the active agent to be incorporated, and removing the solvent from the matrix thereby forming the matrix of the polymer with the active agent distributed throughout the matrix.

### EXAMPLES

### Example 1

### Glucose Normalization in Diabetic ob/ob Mice and Streptozotocin-Nicotinamide Diabetic Minipigs (STZ-NIA-Minipigs)

Method 1

Severely diabetic (morning blood glucose (BG) 20 mM) ob/ob mice (from University of Umeå, Sweden) were treated with vehicle or a liver selective glucokinase activator (GKas), the structure/synthesis of the compounds as described in WO 2004 002481, (160 mg/kg, p.o. at 09:00 and 21:00, formulated as a lysine salt in 0.5% high-viscosity carboxymethyl-cellulose) for 3 weeks. A group of vehicle treated lean non-diabetic mice was also included to measure the degree of normalization of diabetes. Blood glucose (tail vein, conscious animals) was measured every other day, just before dosing in the morning and afternoon, and HbA_{1C} was measured once weekly. At the end of the 3 week study, the mice were anaesthetized; the liver and kidneys were freeze clamped in liquid nitrogen for later determination of glycogen levels (Katz, J., Golden, S, and Wals, P. A. (1976) Proc. Natl. Acad. Sci. U.S.A. 73, 3433-3437).

Method 2

Diabetes/reduced beta-cell mass was induced in adult male Göttingen minipigs (Ellergaard Göttingen Minipigs A/S, Dalmose, Denmark) by dosing Nicotinamide (NIA) (67 mg/kg) and streptozotocin (STZ) (125 mg/kg) at least two weeks before the start of the experiment, as described previously. (reference: Larsen et al. AJP 282:E1342, 2002 “Mild streptozotocin diabetes in the Gottingen minipig: A novel model of moderate insulin deficiency and diabetes”).

An Oral Glucose Tolerance Test (OGTT) is performed before and after induction of diabetes with NIA+STZ.

Blood samples from 18-h fasted animals were taken at −40, −35, −30, −20, −5, 0, 15, 30, 45, 60, 90, 120, 150, 180, 240 minutes and the next day at t=24 hours. At t=−30 the animals are dosed iv with GKas (50 mg/kg) or vehicle in a period of 3 minutes. The oral glucose load was offer as glucose (2 g/kg, 50% solution=4 ml/kg) mixed with 25 g SDS Mini pig fodder at t=0. The glucose is given in a bowl under supervision. Plasma is separated and assayed for glucose, lactate, insulin, C-peptide, glucagon, exposure, intact and total GLP-1 and GIP.

### Example 2

### Prevention of Diabetic Late Complications (e.g. Diabetic Nephropathy) as a Result of Glucose Normalization

Primary prevention of nephropathy relies on good glucose control. For every 1% reduction in HbA_{1C}, the risk of developing microalbuminuria is reduced by 20-40%. Furthermore, in untreated chronically diabetic animals, glycogen accumulates in the renal tubules (diabetic glycogen nephrosis) and this could be an important contribution to renal functional impairment in diabetes. (Bamri-Ezzine S, Ao Z J, Londono I, Gingras D, Bendayan M. LABORATORY INVESTIGATION. 83 (7): 1069-1080 July 2003. Nannipieri M, Lanfranchi A, Santerini D, Catalano C, Van de Werve G, Ferrannini E.)) NEPHRON 87 (1): 50-57 January 2001.)

Glycogen levels in the kidney of vehicle treated diabetic ob/ob mice were more than double (15.5±2.0 μmol/g wW) of lean non-diabetic mice (6.0±0.9 μmol/g wW).

Treatment of with GKas for 3-weeks, normalized kidney glycogen levels (6.3±1.6 μmol/g wW), indicating that the normoglycemia obtained after GK activation will prevent/improved diabetic nephropathy.

### Example 3

### Effect of a Liver Selective GK Activator on Insulin Requirement in Severely Diabetic Göttingen Minipigs

Animals:

Göttingen minipigs are made diabetic using streptozotocin (125 mg/kg)

Methods:

Two days after dosing of streptozotocin, treatment with Insulatard BID is started. Insulin dose is titrated individually to obtain fasting plasma glucose (FPG) between 10 and 15 mM. The starting dose of insulin is 0.2 IU/kg. During the titration period, FPG is measured every 3 days and the insulin dose adjusted.

After three weeks the treatment with GKA or vehicle is started (similar group sizes for the two treatments) and FPG is followed daily. At the same time, the daily dose of insulin is reduced to ¾ of the requirement before treatment in the GKA pigs whereas vehicle pigs are maintained on the same daily dose of insulin. If FPG goes below 10 mM in individual animals, the insulin dose is reduced individually.

Furthermore, animals are subjected to a glucose challenge (2 g/kg glucose) after one week of GKA/vehicle dosing. Daily insulin dose is given in all animals (¾ for GKA and 1 for vehicle pigs)

Blood samples are taken at: −15, −10, −5 min and at ½, 1, 1½, 2, 3, 4, 4½, 5, 6 hours relative to the dosing of GKA/vehicle and glucose. Samples are analysed for plasma human insulin, glucose, porcine C-peptide and glucagon

Interpretation of Results:

If FPG levels and glucose tolerance in the two groups are similar, but the insulin dose is reduced in the GKA group, equal glucose control can be maintained with reduced insulin dosing when using GKA:

### Example 4

### Body Weight Neutrality: Effect of a Liver Selective GKA on Long-Term Body Weight Gain in Rats

Animals:

GK-rats treated for 8 weeks.

Methods:

GK rats are treated with GKA or vehicle for 8 weeks and body weight and food-intake is measured twice weekly during the treatment period. Cumulative food intake and percent increase in body weight during the study are calculated.

Interpretation of the Results:

If the rats treated with the GKA have a body weight increase that is not significantly different from the rats treated with vehicle, treatment with the GKA is body weight neutral.

### Example 5

### Normoglycemia Without Hypoglycemia: Effect of a Liver Selective GKA on Blood Glucose (BG) in Fed and Fasted Diabetic ob/ob Mice

Animals:

Fed (average BG 10-22 mM) or fasted (average BG 8-10 mM) diabetic ob/ob mice.

Methods:

Fed or fasted ob/ob mice are treated with GKA or vehicle and BG is measured before and 2 hours after treatment. The difference in BG before and after treatment is calculated for each animal.

Interpretation of the results:

If, during the BG profile in fed diabetic mice, blood glucose levels will drop by 5-13 mM after treatment with the GKA whilst they will only be reduced by 3 to 6 mM during the BG profile in the fasted diabetic mice where blood glucose levels never will go below 4 mM (normoglycemia), the liver selective GKA has caused normoglycemia without hypoglycemia.

### Example 6

### Less Diabetic KetoAcidosis (DKA): Effect of a Liver Selective GKA on Development of Diabetic Ketoacidosis (DKA) in Severely Diabetic MHBB-Rats

Animals:

Spontaneously diabetic MHBB rats treated with LinPlant insulin.

Methods:

Diabetic MHBB rats are fitted with LinPlant implants according to the manufacturer's instruction. HbA1C is measured and the rats are allocated to treatment groups according to this (average HbA1C will be between 7.1 to 7.6%).

The treatment with GKA or vehicle is started (similar group sizes for the two treatments) and DKA is measured once weekly for 6 weeks by appearance of ketone bodies in urine.

Interpretation of Results:

If the number of animals that present with DKA is decreased in rats treated with the GKA as compared to in the rats treated with vehicle, treatment with the GKA has prevented or delayed the incidence of DKA.

Biological Assays:

Glucokinase Activity Assay (I)

Glucokinase activity is assayed spectrometrically coupled to glucose 6-phosphate dehydrogenase to determine compound activation of glucokinase. The final assay contains 50 mM Hepes, pH 7.1, 50 mM KCl, 5 mM MgCl2, 2 mM dithiothreitol, 0.6 mM NADP, 1 mM ATP, 0.195 μM G-6-P dehydrogenase (from Roche, 127 671), 15 nM recombinant human glucokinase. The glucokinase is human liver glucokinase N-terminally truncated with an N-terminal His-tag ((His)8-VEQILA . . . Q466) and is expressed in E. coli as a soluble protein with enzymatic activity comparable to liver extracted GK.

The purification of His-tagged human glucokinase (hGK) was performed as follows: The cell pellet from 50 ml E. coli culture was resuspended in 5 ml extraction buffer A (25 mM HEPES, pH 8.0, 1 mM MgCl2, 150 mM NaCl, 2 mM mercaptoethanol) with addition of 0.25 mg/ml lysozyme and 50 μg/ml sodium azide. After 5 minutes at room temperature 5 ml of extraction buffer B (1.5 M NaCl, 100 mM CaCl2, 100 mM MgCl2, 0.02 mg/ml DNase 1, protease inhibitor tablet (Complete® 1697498): 1 tablet pr. 20 ml buffer) was added. The extract was then centrifuged at 15,000 g for 30 minutes. The resulting supernatant was loaded on a 1 ml Metal Chelate Affinity Chromatography (MCAC) Column charged with Ni2+. The column is washed with 2 volumes buffer A containing 20 mM imidazole and the bound his-tagged hGK is subsequently eluted using a 20 minute gradient of 20 to 500 mM imididazol in buffer A. Fractions are examined using SDS-gel-electrophoresis, and fractions containing hGK (MW: 52 KDa) are pooled. Finally a gel filtration step is used for final polishing and buffer exchange. hGK containing fractions are loaded onto a Superdex 75 (16/60) gel filtration column and eluted with Buffer B (25 mM HEPES, pH 8.0, 1 mM MgCl2, 150 mM NaCl, 1 mM dithiothreitol). The purified hGK is examined by SDS-gel electrophoresis and MALDI mass spectrometry and finally 20% glycerol is added before freezing. The yield from 50 ml E. coli culture is generally approximately 2-3 mg hGK with a purity >900%.

The compound to be tested is added into the well in final 2.50% DMSO concentration in an amount sufficient to give a desired concentration of compound, for instance 1, 5, 10, 25 or 50 μM. The reaction starts after glucose is added to a final concentration of 2, 5, 10 or 15 mM. The assay uses a 96-well UV plate and the final assay volume used is 200 μl/well. The plate is incubated at 25° C. for 5 min and kinetics is measured at 340 nm in SpectraMax every 30 seconds for 5 minutes. Results for each compound are expressed as the fold activation of the glucokinase activity compared to the activation of the glucokinase enzyme in an assay without compound after having been subtracted from a “blank”, which is without glucokinase enzyme and without compound. The compounds in each of the Examples exhibit activation of glucokinase in this assay. A compound, which at a concentration of at or below 30 μM gives 1.5-fold higher glucokinase activity than the result from the assay without compound, is deemed to be an activator of glucokinase.

The glucose sensitivity of the compounds is measured at a compound concentration of 10 μM and at glucose concentrations of 5 and 15 mM.

Bio1: fold activation @ 5 mM glucose at 10 μM compound concentration,

Bio2: fold activation @ 15 mM glucose at 10 μM compound concentration.

Glucokinase Activity Assay (II)

Determination of Glycogen Deposition in Isolated Rat Hepatocytes:

Hepatocytes are isolated from rats fed ad libitum by a two-step perfusion technique. Cell viability, assessed by trypan blue exclusion, is consistently greater than 800%. Cells are plated onto collagen-coated 96-well plates in basal medium (Medium 199 (5.5 mM glucose) supplemented with 0.1 μM dexamethasone, 100 units/ml penicillin, 100 mg/ml streptomycin, 2 mM L-glutamine and 1 nM insulin) with 4% FCS at a cell density of 30,000 cells/well. The medium is replaced with basal medium 1 hour after initial plating in order to remove dead cells. Medium is changed after 24 hours to basal medium supplemented with 9.5 mM glucose and 10 nM insulin to induce glycogen synthesis, and experiments are performed the next day. The hepatocytes are washed twice with pre-warmed (37° C.) buffer A (117.6 mM NaCl, 5.4 mM KCl, 0.82 mM Mg2SO4, 1.5 mM KH2PO4, 20 mM HEPES, 9 mM NaHCO3, 0.1% w/v HSA, and 2.25 mM CaCl2, pH 7.4 at 37° C.) and incubated in 100 μl buffer A containing 15 mM glucose and increasing concentrations of the test compound, such as for instance 1, 5, 10, 25, 50 or 100 μM, for 180 minutes. Glycogen content is measured using standard procedures (Agius, L. et al, Biochem J. 266, 91-102 (1990). A compound, which when used in this assay gives a significant increase in glycogen content compared to the result from the assay without compound, is deemed to have activity in this assay.

Glucokinase Activity Assay (III)

Stimulation of Insulin Secretion by Glucokinase Activators in INS-1E Cells

The glucose responsive β-cell line INS-1E is cultivated as described by Asfari M et al., Endocrinology, 130, 167-178 (1992). The cells are then seeded into 96-well cell culture plates and grown to a density of approximately 5×104 per well. Stimulation of glucose dependent insulin secretion is tested by incubation for 2 hours in Krebs Ringer Hepes buffer at glucose concentrations from 2.5 to 15 mM with or without addition of glucokinase activating compounds in concentrations of for instance 1, 5, 10, 25, 50 or 100 μM, and the supernatants collected for measurements of insulin concentrations by ELISA (n=4). A compound, which when used in this assay gives a significant increase in insulin secretion in response to glucose compared to the result from the assay without compound, is deemed to have activity in this assay.

In rodent animal models of type 2 diabetes, and in diabetic STZ-NIA minipigs, acute or chronic administration of a liver selective GK activator results in blood glucose normalization without signs of hypoglycemia. Moreover, administration of the same dose to fasted normal and diabetic rats/mice did not induce hypoglycemia.

All references, including publications, patent applications, and patents, cited herein are hereby incorporated by reference to the same extent as if each reference were individually and specifically indicated to be incorporated by reference and were set forth in its entirety herein (to the maximum extent permitted by law).

Any combination of the above-described elements in all possible variations thereof is encompassed by the invention unless otherwise indicated herein or otherwise clearly contradicted by context.

The use of the terms “a” and “an” and “the” and similar referents in the context of describing the invention (especially in the context of the following claims) are to be construed to cover both the singular and the plural, unless otherwise indicated herein or clearly contradicted by context.

The terms “comprising,” “having,” “including,” and “containing” are to be construed as open-ended terms (i.e., meaning “including, but not limited to,”) unless otherwise noted and should be read as encompassing the phrases “consisting”, “substantially comprised of,” and “consisting essentially of” (e.g., where a disclosure of a composition “comprising” a particular ingredient is made, it should be understood that the invention also provides an otherwise identical composition characterized by, in relevant part, consisting essentially of the ingredient and (independently) a composition consisting solely of the ingredient).

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

Preferred embodiments of this invention are described herein. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law.

1. A method for the treatment of glucokinase-deficiency mediated conditions/diseases, or conditions benefiting from an increase in glucokinase activity, comprising administering to a subject in need of such treatment a liver-selective glucokinase activator or pharmaceutical composition thereof, wherein blood glucose normalization occurs with reduced risk of hypoglycemia.

2. A method according to claim 1, wherein the condition/disease is type 1 diabetes.

3. A method according to claim 2, wherein the liver-selective glucokinase activator is administered in combination with insulin, short-acting insulin, long-acting insulin, or a combination thereof.

4. A method according to claim 1, wherein the glucokinase-deficiency mediated condition/disease is caused by a glucokinase mutation.

5. A method according to claim 4, wherein the glucokinase-deficiency mediated condition/disease is Maturity-Onset Diabetes of the Young, Neonatal Diabetes Mellitus, or Persistent Neonatal Diabetes Mellitus.

6. A method for preventing the development of diabetes in subjects exhibiting symptoms of Impaired Glucose Tolerance, Gestational Diabetes Mellitus, Polycystic Ovarian Syndrome, Cushings syndrome or Metabolic Syndrome comprising administering to a subject in need of such treatment a liver-selective glucokinase activator or pharmaceutical composition thereof, wherein blood glucose normalization occurs with reduced risk of hypoglycemia.

7. A method for preventing microvascular diseases comprising administering to a subject in need of such treatment a liver-selective glucokinase activator or pharmaceutical composition thereof.

8. A method for preventing macrovascular diseases in subjects exhibiting symptoms of Impaired Glucose Tolerance, Gestational Diabetes Mellitus, or Metabolic Syndrome, comprising administering to a subject in need of such treatment a liver-selective glucokinase activator or pharmaceutical composition thereof, alone or in combination with lipid-lowering drugs, wherein blood glucose normalization occurs with reduced risk of hypoglycemia.

9. A method for the preservation of beta-cell mass and function comprising administering to a subject in need of such treatment a liver-selective glucokinase activator or pharmaceutical composition thereof, wherein blood glucose normalization occurs with reduced risk of hypoglycemia.

10. A method for preventing amyloid beta peptide induced cell death comprising administering to a subject in need of such treatment a liver-selective glucokinase activator or pharmaceutical composition thereof, wherein blood glucose normalization occurs with reduced risk of hypoglycemia.

11. A method according to claim 1, wherein the subject is a veterinary subject.

12. A method according to claim 11, wherein the liver-selective glucokinase activator is administered as a food additive.

13. A method for the treatment of hepatic conditions benefiting from blood glucose normalization comprising administering to a subject in need of such treatment a liver-selective glucokinase activator or pharmaceutical composition thereof, wherein blood glucose normalization occurs with reduced risk of hypoglycemia.

14. A method for the treatment of hepatic conditions benefiting from improved liver function comprising administering to a subject in need of such treatment a liver-selective glucokinase activator or pharmaceutical composition thereof

15. A method for the treatment of hyperglycemic conditions that result from critical illness, or as a consequence of therapeutic intervention comprising administering to a subject in need of such treatment a liver-selective glucokinase activator or pharmaceutical composition thereof, wherein blood glucose normalization occurs with reduced risk of hypoglycemia.

16. A method for the treatment of hepatic conditions that result from critical illness like cancer, or are a consequence of therapy, for example cancer therapy or HIV-treatment, comprising administering to a subject in need of such treatment a liver-selective glucokinase activator or pharmaceutical composition thereof.

17. A method of treatment adjuvant to insulin in insulin-requiring diabetes type 2, or as replacement for insulin comprising administering to a subject in need of such treatment a liver-selective glucokinase activator or pharmaceutical composition thereof, wherein blood glucose normalization occurs with reduced risk of hypoglycemia.

18. A method for the treatment of lipodistrophy comprising administering to a subject in need of such treatment a liver-selective glucokinase activator or pharmaceutical composition thereof, wherein blood glucose normalization occurs with reduced risk of hypoglycemia.

19. A method for the treatment of hyperglycemia resulting from severe physical stress without signs of liver failure comprising administering to a subject in need of such treatment a liver-selective glucokinase activator or pharmaceutical composition thereof, wherein blood glucose normalization occurs with reduced risk of hypoglycemia.

20. A method according to claim 19, wherein the severe physical stress is multiple trauma, or diabetic ketoacidosis.

21. A method for preventing apoptotic liver damage comprising administering to a subject in need of such treatment a liver-selective glucokinase activator or pharmaceutical composition thereof.

22. A method for preventing hypoglycemia comprising administering to a subject in need of such treatment a liver-selective glucokinase activator or pharmaceutical composition thereof, wherein blood glucose normalization occurs with reduced risk of hypoglycemia.

23. A method for increasing beta-cell mass and function comprising administering to a subject in need of such treatment a liver-selective glucokinase activator or pharmaceutical composition thereof, wherein blood glucose normalization occurs with reduced risk of hypoglycemia.

24. A method of preventing type 1 diabetes comprising administering to a subject in need of such treatment a liver-selective glucokinase activator or pharmaceutical composition thereof, wherein blood glucose normalization occurs with reduced risk of hypoglycemia.

25. A method of preserving and/or increasing beta-cell mass and function in patients having undergone pancreatic islet/beta-cell transplantation comprising administering to a subject in need of such treatment a liver-selective glucokinase activator or pharmaceutical composition thereof.

26. A method of improving glucose control during and after surgery comprising administering to a subject in need of such treatment a liver-selective glucokinase activator or pharmaceutical composition thereof.

27. A method of improving liver function and/or survival in patients undergoing liver transplantation comprising administering to a subject in need of such treatment a liver-selective glucokinase activator or pharmaceutical composition thereof.

28. The method according to claim 27, wherein the administration occurs before, during or after transplantation, or any combination thereof.

29. A method of obtaining blood glucose normalization comprising administering to a subject in need of such treatment a liver-selective glucokinase activator or pharmaceutical composition thereof, wherein blood glucose normalization occurs with reduced risk of hypoglycemia.

30. A method of preventing or ameliorating diabetic late complications comprising administering to a subject in need of such treatment a liver-selective glucokinase activator or pharmaceutical composition thereof.

31. A method of treating type 1 or type 2 diabetes comprising administering to a subject in need of such treatment a liver-selective glucokinase activator or pharmaceutical composition thereof, wherein the treatment does not result in a weight gain.

32. A method of preventing diabetic ketoacidosis comprising administering to a subject in need of such treatment a liver-selective glucokinase activator or pharmaceutical composition thereof.
